(19) United States
(12) Patent Application Publication
(10) Pub. No.: US 2008/0269699 A1
(43) Pub. Date: Oct. 30, 2008

(54) Ostomy pouch

(76) Inventor: Lois Diane O'Toole, Carmel, IN (US)

Correspondence Address: BOWMAN & ASSOCIATES, 1016 3rd Avenue, SW, Suite #106, CARMEL, IN, 46032, US

(21) Appl. No.: 11/789,265

(22) Filed: Apr. 24, 2007

(51) Int. Cl. A61F 5/445

(52) U.S. Cl. 604/332

(57) ABSTRACT

An ostomy pouch is formed to conceal, obscure, hide, cloak, or otherwise prevent the viewing of contents of the ostomy pouch except for a “sight glass” or window formed integral with the pouch and positioned therein for viewing at least a portion of a stoma receptor of the ostomy pouch. In one embodiment, the ostomy pouch is formed by an opaque plastic bag incorporating a stoma receptor in one side of the bag and a transparent window in another side of the bag. The transparent window is preferably, but not necessarily, positioned opposite and adjacent the stoma receptor. The ostomy pouch may be entirely sealed except for a stoma receptor orifice or may be open at one end for emptying its contents and incorporate or use a closure at the open end.

### BACKGROUND OF THE INVENTION

1. Field of the Invention

The present invention relates to ostomy appliances and, in particular, to an ostomy bag or pouch used for the collection of waste material.

2. Background Information

Persons may undergo major abdominal surgery, known as an ostomy, whose normal bowel function, bladder function and/or different parts of the gastrointestinal and urinary tract are or have become impaired due to a birth defect, injury, disease or other disorder. Ostomy surgery often involves removal or bypass of impaired or diseased tissue and the creation of a hole in the abdominal wall of the patient through which a section of the bowel or bladder extends. The portion of the protruding tissue (e.g. the intestine or bladder) is referred to as a stoma or abdominal stoma. The three most common types of abdominal stoma result from and may be classified as a colostomy, ileostomy or urostomy (“ostomies”). Bowel attachment surgery is termed a ileostomy in which the small intestine is involved and is termed a colostomy when the large intestine is involved. A urostomy involves the bladder.

In all ostomies the patient is unable to control the passage of bodily waste material from the stoma and thus involuntarily discharges such waste material from the stoma. Because of this, the waste material is collected by an ostomy pouch that is positioned over the stoma and attached to the user's abdomen. Users whose discharged is of a solid consistency usually employ a disposable pouch. The term disposable means that the pouch is disposed of after a single use. Users whose discharge is of a liquid consistency usually employ a collecting pouch having a bottom opening that permits the contents to be emptied and the pouch reused for a period of time. Such pouches include some type of closure that seals the bottom opening while the pouch is collecting the waste material. Various types of closure systems have been developed and patented (see, e.g. U.S. Pat. No. 4,403,991 and the discussion therein).

Attachment of the ostomy appliance to the ostomy patient is achieved with either a one piece system or a two piece system. In the two piece system, a flange is adhered to the body of the ostomy patient proximate the stoma and a bag device for waste collection. The pouch is mechanically coupled to the flange which allows the bag to be removably fastened to the flange and be in communication with the stoma. In the one piece system, both the means of adhering the bag to the stoma and the body and the bag are integral. Current adhesive systems provide an inner layer over which a protective paper or cloth tape is adhered to provide a barrier through which waste material will not pass but that allows oxygen and water vapor to pass.

In both cases, the ostomy bag or pouch is made from plastic such as polyvinyl chloride (PVC). The bags are typically formed from layers of plastic. Because ostomy bags hold human waste material, it is desirable from everyone's perspective to obscure the contents of a user's ostomy bag and/or otherwise shield its contents from view by both the user and a third party. This has heretofore been accomplished by using opaque and/or colored plastic for the pouch. A woven cotton layer may then be provided over the plastic. Since the plastic pouch is opaque or colored, neither the user nor a third party can see the contents of the pouch. Another manner of concealing the contents of the ostomy pouch and one which provides a more aesthetically pleasing appearance is ostomy bags made of opaque or colored plastic in various configurations or iconic shapes to disguise the function of the bag. This is exemplified in U.S. Patent Publication 2005/0261645 by Conrad et al., wherein ostomy bags are disclosed having an iconic shape such as a teddy bear, a purse, a heart, a butterfly, a seashell, a dog, a cat, or the like.

While the above prior art ostomy bags perform their intended function of hiding, concealing and/or obscuring their contents, they also hide, conceal and/or obscure the bag's stoma opening and/or flange assembly used to attach the ostomy bag to the user's stoma. Thus, the bag's stoma opening cannot be seen through the bag. For users that need to be able to see the bag's stoma opening in order to easily register the bag's stoma opening with the user's stoma/stoma flange during installation, this creates a problem. Since the bag's stoma opening cannot be seen through the bag, it makes it quite difficult to properly attach the bag when the ostomy bag's stoma opening cannot be seen from the reverse side as when looking in a mirror in order to attach the ostomy bag. This makes a user tend toward using the transparent ostomy bags. Thus, however, sacrifices discreetness for ease of use.

It is thus apparent from the above that here there is a need for a discreet type of ostomy bag or pouch that is also easy to attach to a user's stoma.

### SUMMARY OF THE INVENTION

The present invention is an ostomy bag, pouch or appliance formed to conceal, obscure, hide, cloak, or otherwise prevent the viewing of contents thereof except for a transparent window formed in the pouch and positioned for viewing at least a portion of a stoma receptor of the ostomy pouch. In one embodiment, the ostomy pouch is formed by an opaque plastic bag incorporating a stoma receptor in one side of the bag and a transparent window in another side of the bag. The transparent window is preferably, but not necessarily, positioned opposite and adjacent the stoma receptor. The ostomy pouch may be entirely sealed except for a stoma receptor orifice or may be open at one end for emptying its contents and incorporate or use a closure at the open end.

The present ostomy pouch thus provides a discreet ostomy appliance that still allows for easy attachment by giving the user a manner of viewing attachment features of the ostomy appliance through a front side while the remainder of the appliance is opaque.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a front plan view of an ostomy pouch fashioned in accordance with the principles of the present invention;

FIG. 2 is a back plan view of the ostomy pouch of FIG. 1;

FIG. 3 is a sectional view of the ostomy pouch of FIG. 1 taken along line 3-3 of FIG. 2;

FIG. 4 is an enlarged top side perspective view of a stoma receptor of the ostomy pouch of FIG. 1;

FIG. 5 is an enlarged top side perspective view of an alternative embodiment of a stoma receptor for an ostomy pouch per the present principles; and

FIG. 6 is an enlarged bottom side perspective view of a stoma receptor of the present ostomy pouch.

Like reference numerals indicate the same or similar parts throughout the several figures.

An overview of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DESCRIPTION OF THE PREFERRED EMBODIMENT

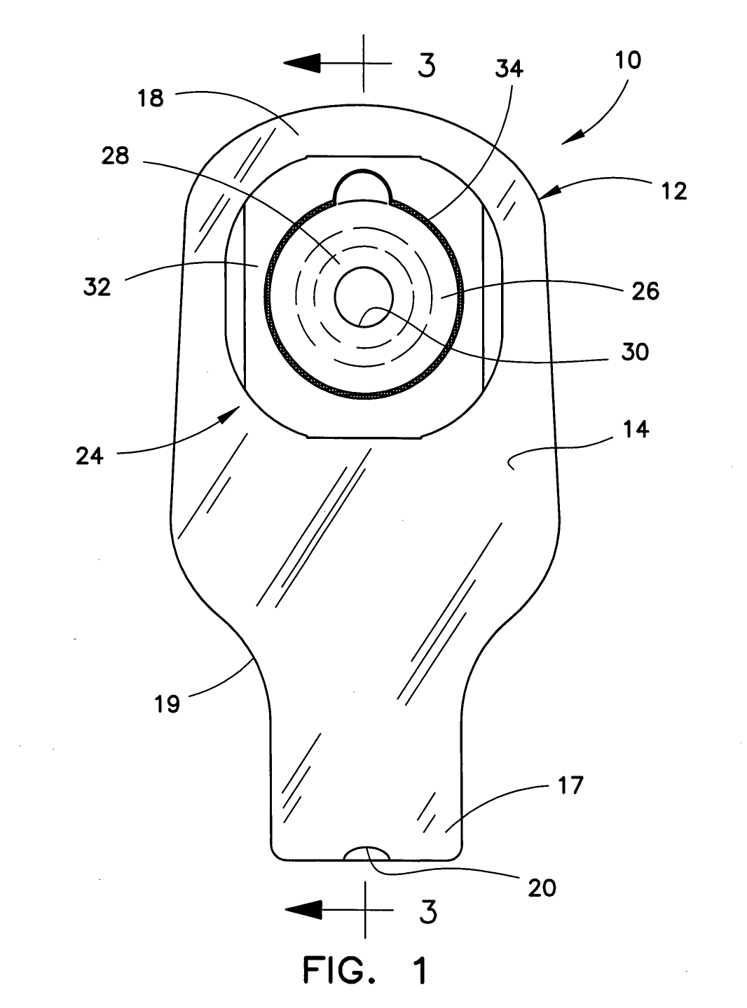

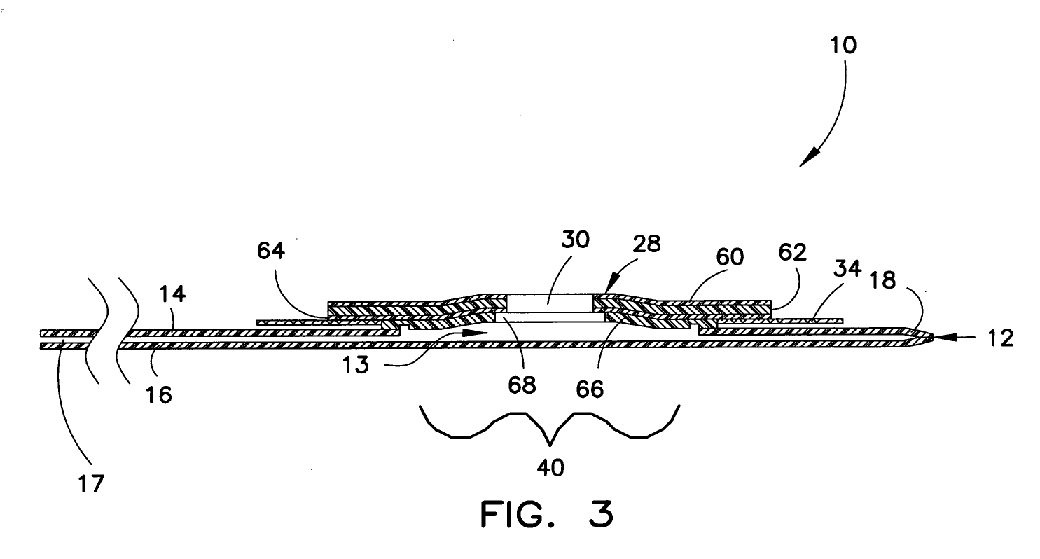

Referring to FIGS. 1-5 there is depicted an exemplary embodiment of an ostomy pouch, appliance or bag (collectively, pouch), generally designated 10, fashioned in accordance with the principles of the present invention as described herein, particularly for use with a stoma and for the collection of human waste material as a result of an ostomy. The ostomy pouch 10 may be fashioned as a drainable pouch or a collection-only pouch and is preferably, but not necessarily, a disposable type ostomy pouch. The ostomy pouch 10 is formed by a preferably plastic body 12 defining a bag or pouch, the plastic being one that is known in the art for manufacturing ostomy bags. Other materials may be used. The body 12 is formed by a first side 14 and a second side 16 lying over, opposite of and/or adjacent to the first side 14. The first and second sides 14, 16 may be separate pieces or unitary and formed of one or more layers as appropriate. The sides are formed of plastic as is known in the art and of an opaque plastic such that the contents of the pouch are obscured. The plastic may also be colored or otherwise marked so as to hide, obscure, mask, blur, or block out the contents of the pouch. The term opaque as used herein encompasses all such manners/markings. As best discerned in FIG. 3, the first and second sides 14 and 16 are sealed or joined together at one end 18 so as to form an interior 13 for the collection of waste material. The first and second sides 14, 16 are shaped so as to form a tapered section or neck 19 that is distal the end 18. The neck 19 terminates in end 17. In the embodiment shown, the end 17 is open (i.e. the first and second sides 14, 16 are not joined or sealed) and includes a cutout 20. Since the end 17 is open, the end 17 is shaped and/or configured to receive a closure device (not shown) for temporarily closing or sealing the end opening 17. Alternatively, the end 17 may include an integral closure or closure device such as is known in the art.

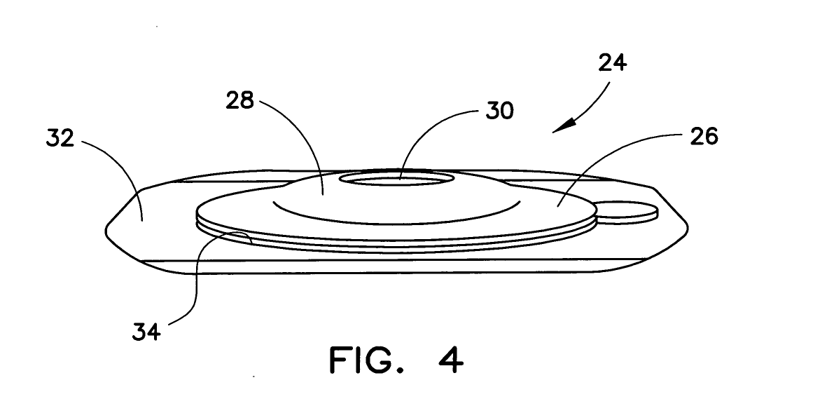

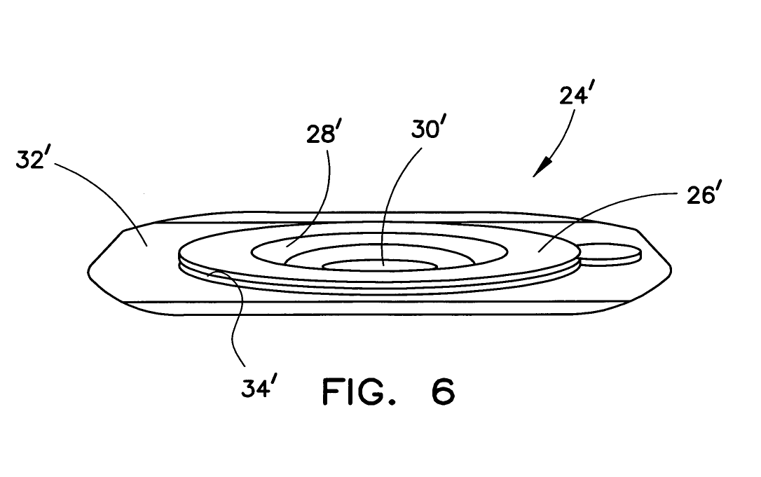

The first side 12 is the side of the pouch 10 that is positioned inwardly toward the user or next to the user's skin when the pouch is installed onto the user's stoma (not shown). As such, the first side 12 has a stoma receptor 24 that is configured to be received onto and about the user's stoma/stoma area and adhered thereto such as is known in the art. The stoma receptor 24 is the manner of attachment and communication with the user's stoma. The stoma receptor 24 includes a generally annular convex base portion 26 terminating in a conical convex nozzle 28 having an orifice or opening 30. The opening 30 is typically round. The stoma receptor 24 may thus be termed a convex stoma receptor and this is particularly seen in FIG. 4. The convex stoma receptor 24 is used on/with a corresponding stoma. It should be appreciated that the stoma receptor may take different forms. FIG. 6 depicts an alternative embodiment of a stoma receptor 24′. The stoma receptor 24′ is likewise configured to be received onto and about the user's stoma/stoma area and adhered thereto such as is known in the art. The stoma receptor 24′ includes a generally annular concave base portion 26′ terminating in a conical concave nozzle 28′ having an orifice or opening 30′. The stoma receptor 24′ may thus be termed a concave stoma receptor. The concave stoma receptor 24′ is used on/with a corresponding stoma. Other configurations are contemplated.

As best seen in FIG. 3, the base portion 26 is formed of multiple plies of material. Particularly, the base portion 26 has an outer covering layer 60 (e.g. paper) that is overlaid onto a cushioning/cushiony material layer 62. A layer of adhesive is provided on the outside surface of the cushion layer 62. The outer covering layer 60 is removed for use thereby exposing the adhesive layer. Below the cushion layer 62 is a transition layer 64 that may be made of a fibrous material. Below the transition layer 64 is the mesh layer 34. The mesh layer 34 extends over and is interposed between the plastic pouch 12 and adhesion layers of the stoma receptor 24. A rigid (e.g. plastic) coupling ring 66 is provided between the mesh layer 34 and the plastic pouch 12. The coupling ring 66 includes a round opening 68 corresponding to opening 30 (and may be considered as one) and provides sealing to and between the stoma receptor and the plastic pouch such that the opening 30 is in communication with the interior 13 of the pouch 12. Of course, it should be appreciated that the configuration, structure and/or materials of the stoma receptor may be different than that described and/or modified as desired. Moreover, the manner of communication, adhesion and/or coupling of the stoma receptor/pouch may be different than that described and/or modified as desired.

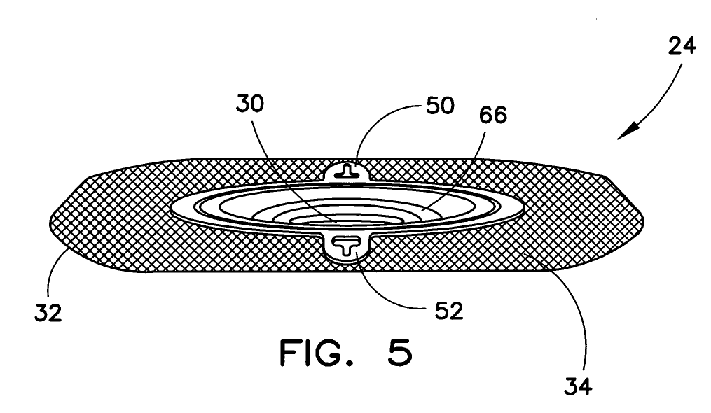

The exposed portion of the stoma receptor 24 (the portion that is adjacent to the user's skin) is particularly shown in FIG. 4. FIG. 5 shows a backside view of the stoma receptor 24. Particularly, the mesh layer 34 is shown which is between the plastic pouch 12 and the plastic coupling ring 66. The coupling ring 66 includes coupling flanges 50 and 52 that are used to attach the ostomy appliance 10 to the user. Of course, other configurations may be used.

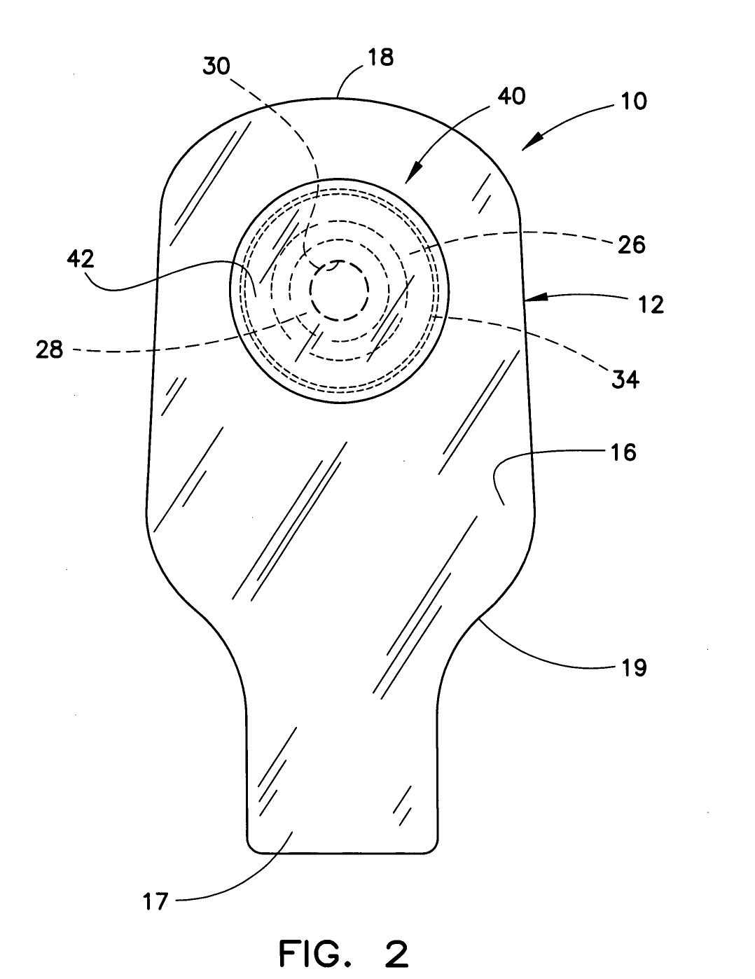

Referring now to FIG. 2, the second side 16 of the pouch 10 is shown. The second side 16 of the pouch is the side that faces outwardly from the user when installed. As indicated above, the pouch is made from an opaque plastic so that the second side 16 is opaque as termed herein. However, in accordance with the principles of the present invention, the second side 16 includes a window, porthole or the like 40 that is situated opposite the stoma receptor 24 and particularly the nozzle 28 and preferably, but not necessarily, formed integral with the second side 14. The window 40 is formed by a transparent plastic portion 42 such that appropriate portions of the stoma receptor 24 (e.g. the nozzle and the opening 30) can be seen/viewed from the second side 16 (which may be considered the front side of the pouch when worn by a user, which makes the first side 14 the back side when worn by a user). The size and/or configuration of the window 40 may be modified as desired. The window 40 allows the user to see the stoma opening of the pouch 10 for easier attachment of the pouch 10 to the user. Since the remainder of the pouch 10 is made from an opaque plastic, the majority of the contents of the pouch 10 is obscured, hidden, masked, blurred, or blocked out from external view.

It should be appreciated that the present ostomy pouch may be fashioned in other manners, shapes, perimeter shapes, iconic shapes, configurations and/or the like while incorporating a window of a transparent material on a front side of the pouch for viewing stoma attachment features on the front side for aligning and/or proper attachment of the pouch by the user to the user. The window may have a pattern itself or other markings as long as they provide at least a limited view of the stoma attachment features on the front side of the pouch. Moreover, colors, fabrics, overlays and/or the like may be used.

While the invention has been illustrated and described in detail in the drawings and foregoing description, the same is to be considered as illustrative and not restrictive in character, it being understood that only a preferred embodiment has been shown and described and that all changes and modifications that come within the spirit of the invention are desired to be protected.

What is claimed is:

1. An ostomy pouch comprising:
an opaque plastic bag defining an interior for the collection of waste material;
a stoma receptor formed in one side of the opaque plastic bag and having a stoma orifice in communication with the interior; and
a window formed in another side of the opaque plastic bag and positioned to allow a user to view at least a portion of the stoma orifice therethrough.

2. The ostomy pouch of claim 1, wherein the window is formed opposite the stoma orifice.

3. The ostomy pouch of claim 1, wherein the window is formed integral with the another side of the opaque plastic bag.

4. The ostomy pouch of claim 1, wherein the opaque plastic bag is formed as one piece.

5. The ostomy pouch of claim 1, wherein the opaque plastic bag is formed from several pieces.

6. The ostomy pouch of claim 1, wherein the opaque plastic bag includes a drainage opening.

7. An ostomy appliance comprising:
a pouch made from an opaque plastic and defining first and second sides and an interior;
a stoma receptor situated on the first side of the pouch, the stoma receptor having a stoma opening that is configured to receive a stoma of a user and is in communication with the interior; and
a transparent plastic porthole formed in the second side of the opaque plastic pouch opposite the stoma opening and allowing viewing thereof therethrough.

8. The ostomy appliance of claim 7, wherein the pouch is formed as an integral piece of plastic.

9. The ostomy appliance of claim 7, wherein the stoma receptor includes an adhesive cushioned layer configured to be received on the user.

10. The ostomy appliance of claim 7, wherein the transparent plastic porthole is formed integral with the pouch.

11. The ostomy appliance of claim 7, wherein the transparent plastic porthole is formed separate from the pouch.

12. The ostomy appliance of claim 7, wherein the opaque plastic pouch includes a drainage opening.
